(19) United States
(12) Patent Application Publication
(10) Pub. No.: US 2017/0095463 A1
(43) Pub. Date: Apr. 6, 2017

(54) COMBINATION OF PHOSPHATIDYLINOSITOL-3-KINASE (PI3K) INHIBITOR AND A MTOR INHIBITOR

(71) Applicants: Christine Fritsch, Steinbach (FR); Carlos Garcia-Echeverria, Saint-Cloud (FR); Xizhong Huang, Southborough, MA (US); Sauveur-Michel Maira, Habsheim (FR)

(72) Inventors: Christine Fritsch, Steinbach (FR); Carlos Garcia-Echeverria, Saint-Cloud (FR); Xizhong Huang, Southborough, MA (US); Sauveur-Michel Maira, Habsheim (FR)

(73) Assignee: Novartis AG, Basel (CH)

(21) Appl. No.: 15/335,957

(22) Filed: Oct. 27, 2016

Related U.S. Application Data
(63) Continuation of application No. 14/113,316, filed on Oct. 22, 2013.
(60) Provisional application No. 61/478,572, filed on Apr. 25, 2011.

(51) Int. Cl. A61K 31/4439; A61K 45/06; A61K 31/436

(52) U.S. Cl. CPC A61K 31/4439; A61K 31/436; A61K 45/06

(57) ABSTRACT

The present invention relates to a pharmaceutical combination comprising a phosphatidylinositol-3-kinase (PI3K) inhibitor compound which is a 2-carboxamide cycloamino urea derivative or a pharmaceutically acceptable salt thereof and at least one mammalian target of rapamycin (mTOR) inhibitor or a pharmaceutically acceptable salt thereof; a pharmaceutical composition comprising such a combination; and the uses of such a combination in the treatment proliferative diseases, more specifically of mammalian target of rapamycin (mTOR) kinase dependent diseases.

### FIELD OF THE INVENTION

The present invention relates to a pharmaceutical combination comprising a phosphatidylinositol-3-kinase (PI3K) inhibitor compound which is a 2-carboxamide cydoamino urea derivative or a pharmaceutically acceptable salt thereof and at least one mammalian target of rapamycin (mTOR) inhibitor or a pharmaceutically acceptable salt thereof; a pharmaceutical composition comprising such a combination; and the uses of such a combination in the treatment proliferative diseases, more specifically of mammalian target of rapamycin (mTOR) kinase dependent diseases.

### BACKGROUND OF THE INVENTION

It has been shown that mammalian target of rapamycin (mTOR) inhibition can induce upstream insulin-like growth factor 1 receptor (IGF-1R) signaling resulting in AKT activation in cancer cells. This phenomenon has been suggested to play a role in the attenuation of cellular responses to mTOR inhibition and may attenuate the clinical activity of mTOR inhibitors. Increase in pAKT has for instance been found in approximately 50% in the tumours of all patients in a Phase I study in patients with advanced solid tumours (Taberno et al., Journal of Clinical Oncology, 26 (2008), pp 1603-1610).

In spite of numerous treatment options for proliferative disease patients, there remains a need for effective and safe therapeutic agents and a need for their preferential use in combination therapy. The compounds of formula (A), as set forth herein and including (S)-pyrrolidine-1,2-dicarboxylic acid 2-amide 1-(4-methyl-5-[2-(2,2,2-trifluoro-1,1-dimethyl-ethyl)-pyridin-4-yl]-thiazol-2-yl)-amide, are highly selective inhibitors of alpha isoform of the phosphatidylinositol 3-kinase (PI3K). It has been surprisingly discovered that the combination of an effective amount of the alpha-specific PI3K inhibitor compounds of formula (A) with an effective amount of at least one mTOR inhibitor results in unexpected synergistic improvement in the treatment of mammalian target of rapamycin (mTOR) dependent diseases, particularly cancer. When administered simultaneously, sequentially or separately, this alpha-specific PI3K inhibitor compound and the mTOR inhibitor of the present invention interact to strongly inhibit cell proliferation. This beneficial interaction allows reduction in the dose required for each compound, leading to a reduction in the side effects and enhancement of the long-term clinical effectively of the compounds in treatment.

### SUMMARY OF THE INVENTION

It has been now been found in accordance with the present invention that an alpha-isoform specific phosphatidylinositol 3-kinase (PI3K) inhibitor compound of formula (A) or a pharmaceutically acceptable salt thereof reduces or blocks the phosphorylation and activation of AKT by mTOR inhibitors. Accordingly, the present invention relates to a pharmaceutical combination comprising a compound of formula (A) or a pharmaceutically acceptable salt thereof and at least one mTOR inhibitor or a pharmaceutically acceptable salt thereof.

In a preferred embodiment, the compound of formula (A) in the present invention is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”).

In a preferred embodiment, the mTOR inhibitor in the present invention is selected from RAD rapamycin (sirolimus) and derivatives/analogs thereof such as everolimus (RAD001), temsirolimus (CCI-779), zotarolimus (ABT578), SAR543, ascomycin (an ethyl analog of FK506), deferolimus (AP235731 MK-8669), AP23841, KU-0063794, INK-128, EX2044, EX3855, EX7518, AZD08055, OSI-027, WYE-125132, XL765, NV-128, WYE-125132, and EM101/LY303511.

In one aspect, the present invention provides a pharmaceutical combination comprising a compound of formula (A) or a pharmaceutically acceptable salt thereof and at least one mTOR inhibitor or a pharmaceutically acceptable salt thereof for use in treating or preventing an mTOR kinase dependent disease.

In a further aspect, the present invention provides the use of a compound of formula (A) or a pharmaceutically acceptable salt thereof and at least one mTOR inhibitor or a pharmaceutically acceptable salt thereof for the manufacture of a medicament for the treatment or prevention of an mTOR kinase dependent disease.

In further aspect the present invention provides a method of treating or preventing an mTOR kinase dependent disease by administering a compound of formula (A) or a pharmaceutically acceptable salt thereof and at least one mTOR inhibitor or a pharmaceutically acceptable salt thereof.

In a further aspect, the present invention provides a combination of a compound of formula (A) and at least one mTOR inhibitor selected from the group consisting of RAD rapamycin (sirolimus) and derivatives/analogs thereof such as everolimus (RAD001), temsirolimus (CCI-779), zotarolimus (ABT578), SAR543, ascomycin (an ethyl analog of FK506), deferolimus (AP23573/MK-8669), AP23841, KU-0063794, INK-128, EX2044, EX3855, EX7518, AZD08055, OSI-027, WYE-125132, XL765, NV-128, WYE-125132, and EM101/LY303511, wherein the active ingredients are present in each case in free form or in the form of a pharmaceutically acceptable salt, and optionally at least one pharmaceutically acceptable carrier, for simultaneous, separate or sequential use for the treatment of mammalian target of rapamycin (mTOR) kinase dependent diseases.

In a further aspect, the present invention provides a method to reduce or block the phosphorylation and activation of AKT by mTOR inhibitors comprising administering a compound of formula (A) or a pharmaceutically acceptable salt thereof to a warm-blooded animal in need thereof.

In another embodiment, the present invention provides a method of treating a proliferative disease dependent on acquired phosphorylation and activation of AKT during treatment with at least one mTOR inhibitor or a pharmaceutically acceptable salt thereof comprising administering a therapeutically effective amount of a compound of formula (A) or a pharmaceutically acceptable salt thereof to a warm-blooded animal in need thereof.

In further embodiment, the present invention relates to a method of treating a proliferative disease which has become resistant or has a decreased sensitivity to the treatment with at least one mTOR inhibitor or a pharmaceutically acceptable salt thereof comprising administering a therapeutically effective amount of a compound of formula (A) or a pharmaceutically acceptable salt thereof to a warm-blooded animal in need thereof. The resistance is e.g. due to phosphorylation and activation of AKT.

In a further aspect the present invention provides a method for improving efficacy of the treatment of a proliferative disease with at least one mTOR inhibitor or a pharmaceutically acceptable salt thereof comprising administering a combination comprising a compound of formula (A) or a pharmaceutically acceptable salt thereof and at least one mTOR inhibitor or a pharmaceutically acceptable salt thereof to a warm-blooded animal in need thereof.

In one aspect the present invention provides a pharmaceutical composition comprising a PI3K inhibitor compound of formula (A) or a pharmaceutically acceptable salt thereof and at least one mTOR inhibitor or a pharmaceutically acceptable salt thereof.

### DETAILED DESCRIPTION OF THE FIGURES

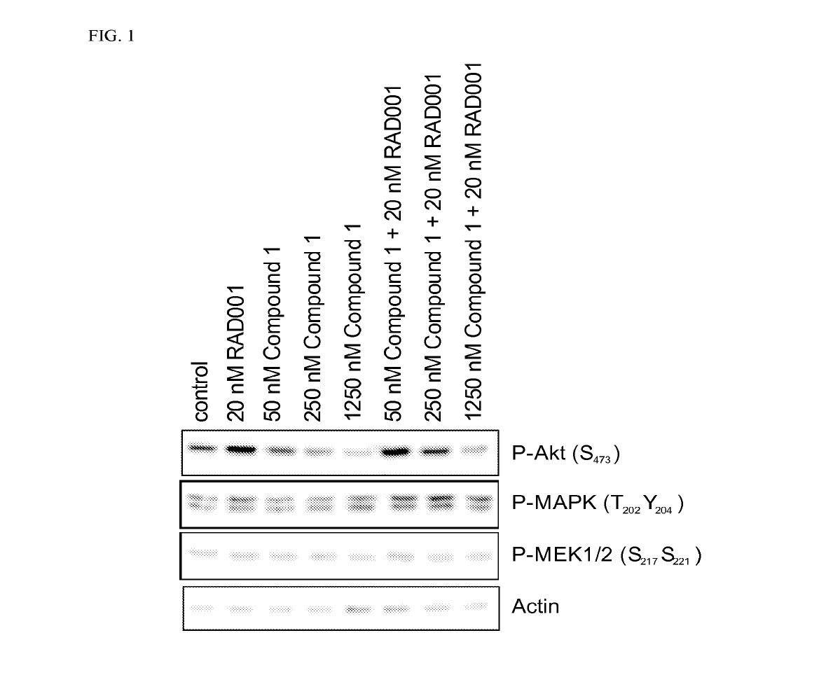

FIG. 1 shows the phosphorylation levels of AKT (S473); MAPK (T202/Y204); MEK1/2 (S217/S221) and actin levels in presence of everolimus (RAD001) single agent, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) single agent, and everolimus (RAD001) in combination with Compound I in BT474 breast tumor cells as detected by Western blot analysis.

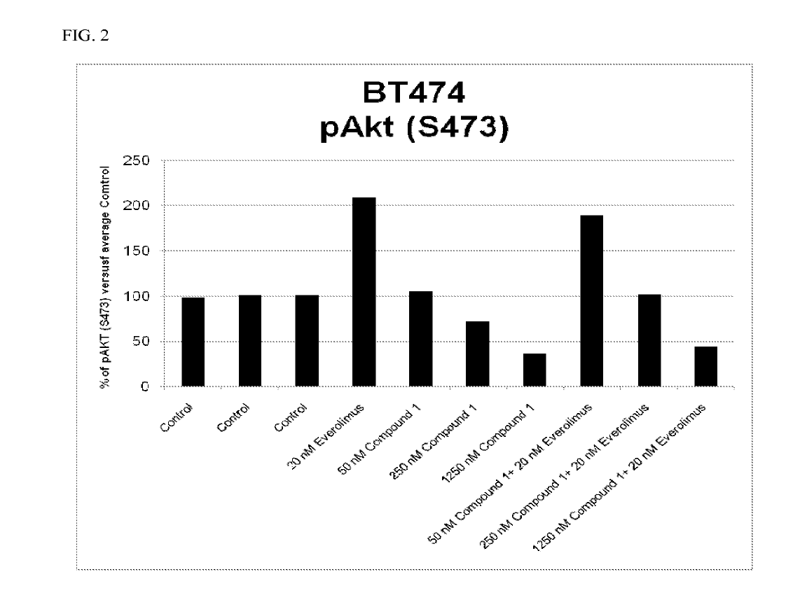

FIG. 2 shows the AKT (S473) phosphorylation levels in presence of everolimus (RAD001) single agent, Compound I single agent and everolimus (RAD001) in combination with Compound I in comparison to the vehicle control in BT474 breast tumor cells as quantified by Reverse Protein Array methodology.

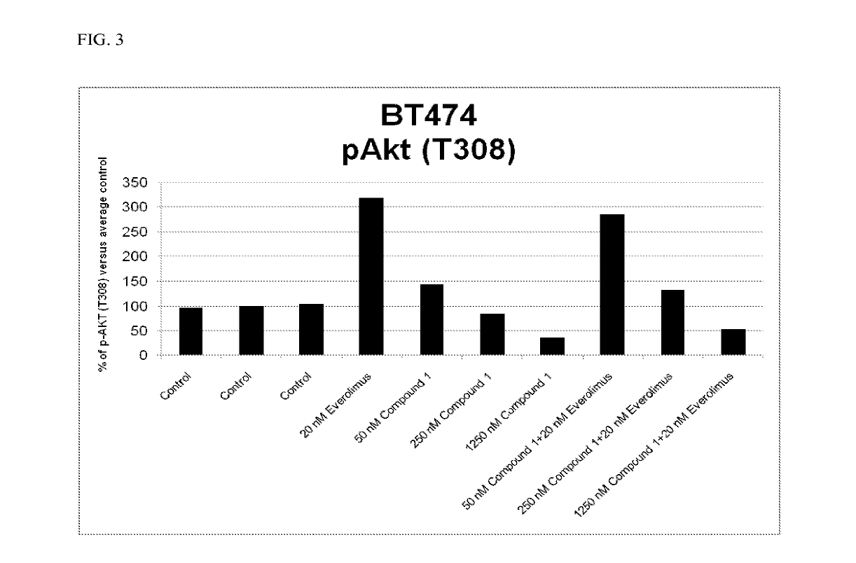

FIG. 3 shows the AKT (T308) phosphorylation levels in presence of everolimus (RAD001) single agent, Compound I single agent and everolimus (RAD001) in combination with Compound I in comparison to the vehicle control in BT474 breast tumor cells as quantified by Reverse Protein Array methodology.

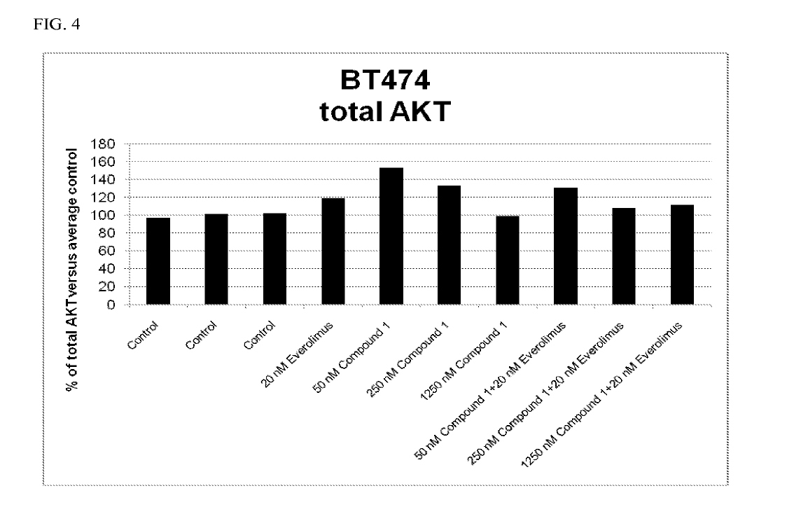

FIG. 4 shows the total AKT expression levels in presence of everolimus (RAD001) single agent, Compound I single agent, and everolimus (RAD001) in combination with Compound I in comparison to the vehicle control in BT474 breast tumor cells as quantified by Reverse Protein Array methodology.

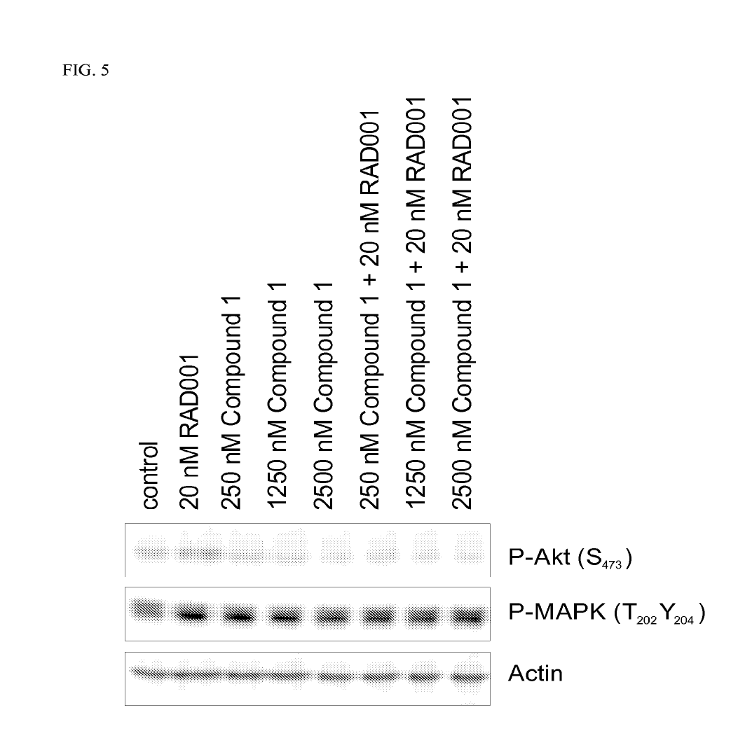

FIG. 5 shows the phosphorylation levels of AKT (S473); MAPK (T202/Y204) and actin levels in presence of everolimus (RAD001) single agent, Compound I single agent, and everolimus (RAD001) in combination with Compound I in MDA-MB231 breast tumor cells as detected by Western blot analysis.

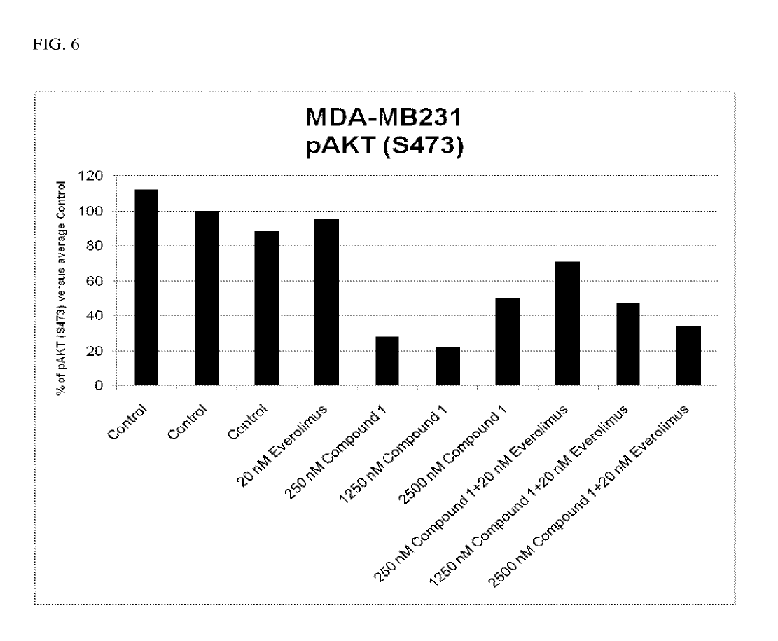

FIG. 6 shows the AKT (S473) phosphorylation levels in presence of everolimus (RAD001) single agent, Compound I single agent, and everolimus (RAD001) in combination with Compound I in comparison to the vehicle control in MDA-MB231 breast tumor cells as quantified by Reverse Protein Array methodology.

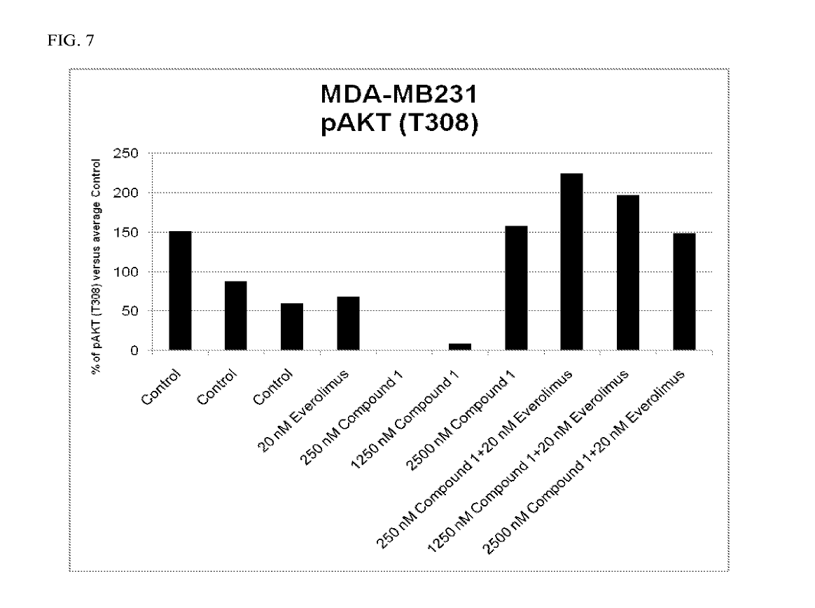

FIG. 7 shows the AKT (T308) phosphorylation levels in presence of everolimus (RAD001) single agent, Compound I single agent, and everolimus (RAD001) in combination with Compound I in comparison to the vehicle control in MDA-MB231 breast tumor cells as quantified by Reverse Protein Array methodology.

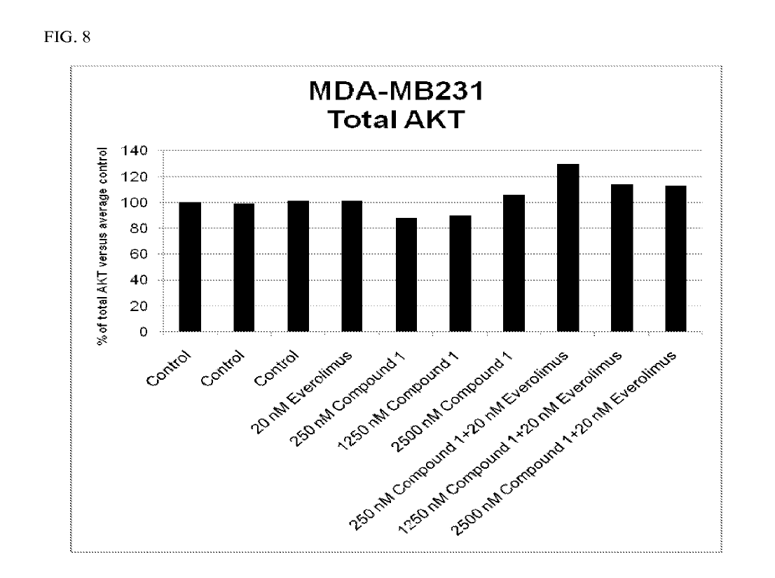

FIG. 8 shows the total AKT expression levels in presence of everolimus (RAD001) single agent, Compound I single agent, and everolimus. (RAD001) in combination with Compound I in comparison to the vehicle control in MDA-MB231 breast tumor cells as quantified by Reverse Protein Array methodology.

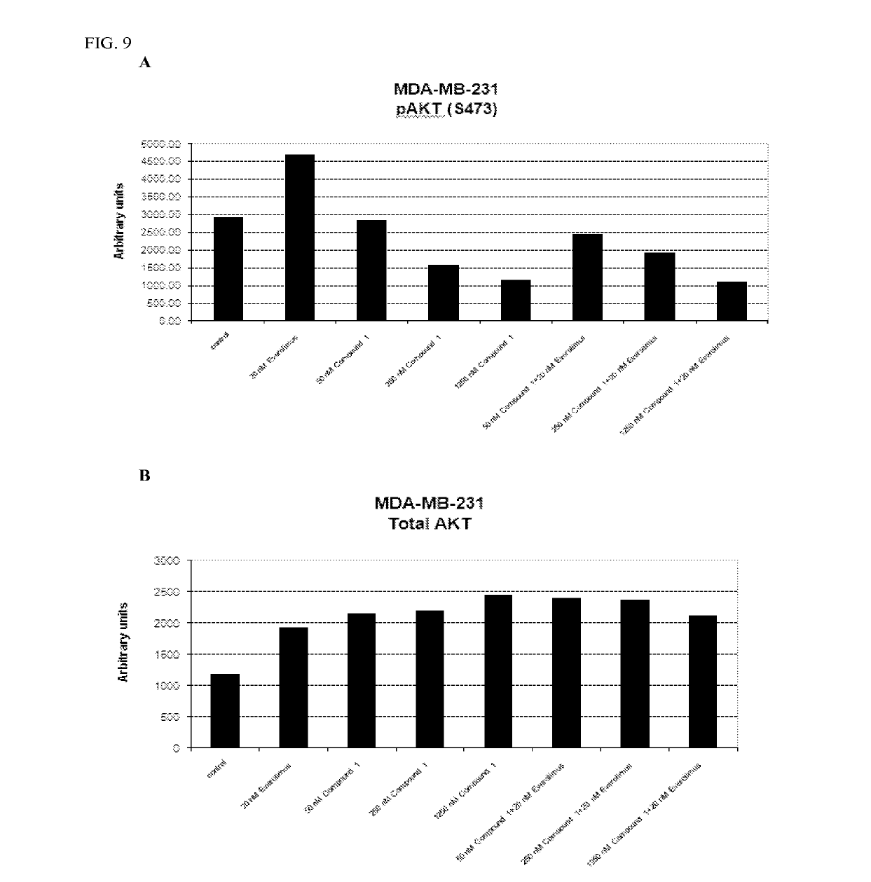

FIG. 9 shows the phosphorylation levels of AKT (S473) (Panel A) and the total levels of AKT (Panel B) in presence of everolimus (RAD001) and everolimus (RAD001) in combination with Compound I in MDA-MB231 breast tumor cells as detected by Western blot and further quantified using the Quantity One software, in a second set of experiment.

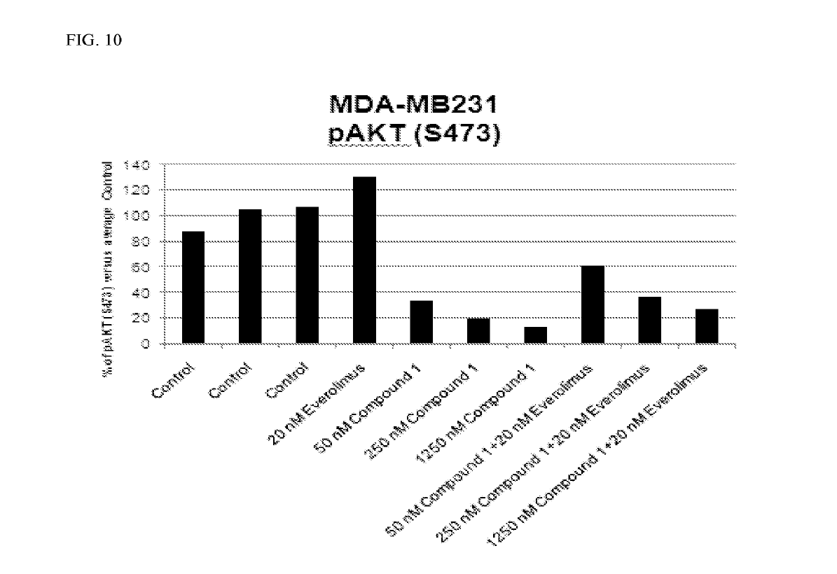

FIG. 10 shows the AKT (S473) phosphorylation levels in presence of everolimus (RAD001) single agent, Compound I single agent, and everolimus (RAD001) in combination with Compound I in comparison to the vehicle control in MDA-MB231 breast tumor cells as quantified by Reverse Protein Array methodology, in a second set of experiment.

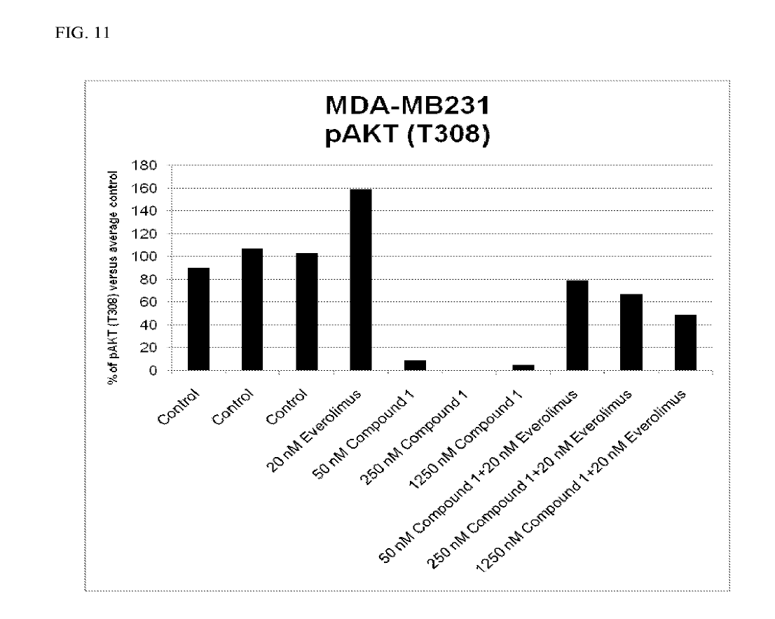

FIG. 11 shows the AKT (T308) phosphorylation levels in presence of everolimus (RAD001) single agent, Compound I single agent, and everolimus (RAD001) in combination with Compound I in comparison to the vehicle control in MDA-MB231 breast tumor cells as quantified by Reverse Protein Array methodology, in a second set of experiment.

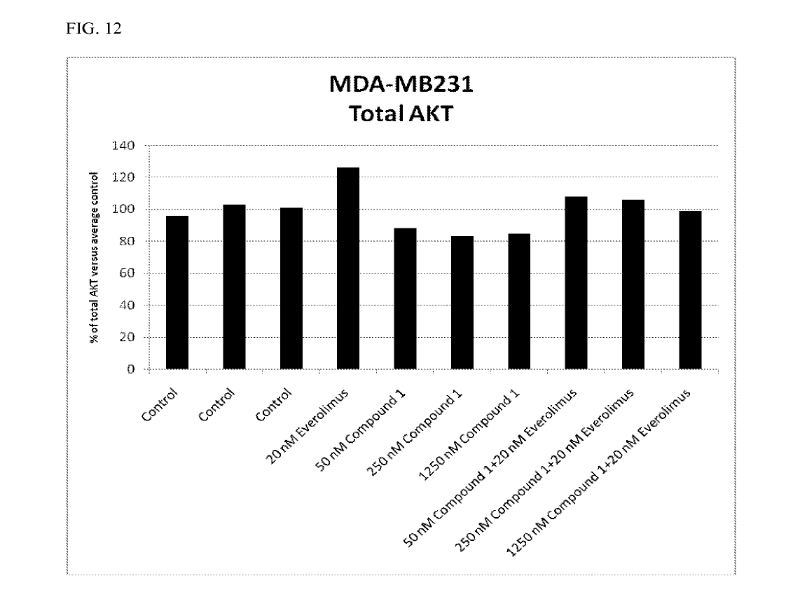

FIG. 12 shows the total AKT expression levels in presence of everolimus (RAD001) single agent, Compound I single agent, and everolimus (RAD001) in combination with Compound I in comparison to the vehicle control in MDA-MB231 breast tumor cells as quantified by Reverse Protein Array methodology, in a second set of experiment.

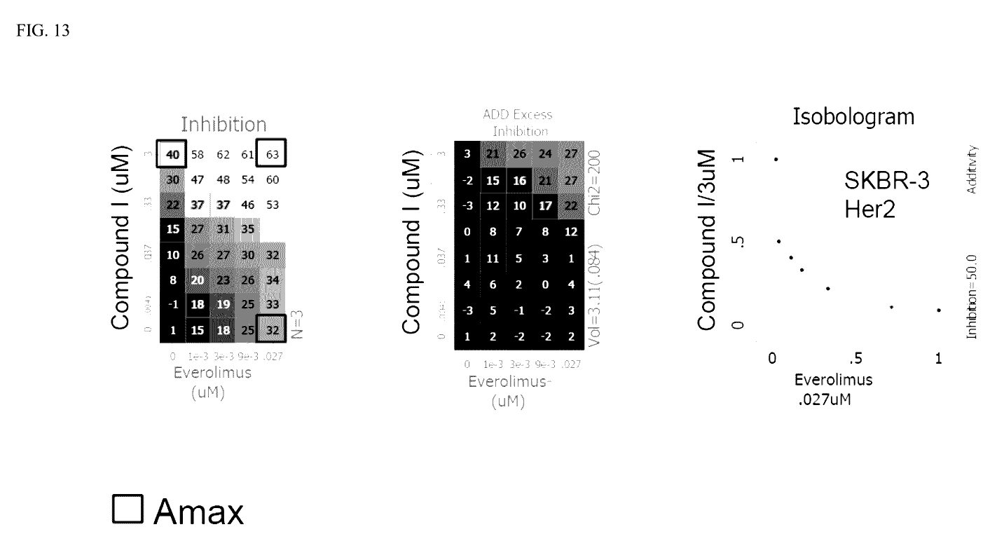

FIG. 13 shows full dose matrix cell proliferation data from single agent and concomitant everolimus (RAD001) and/or Compound I treatment in SKBR-3 human breast cancer cell models.

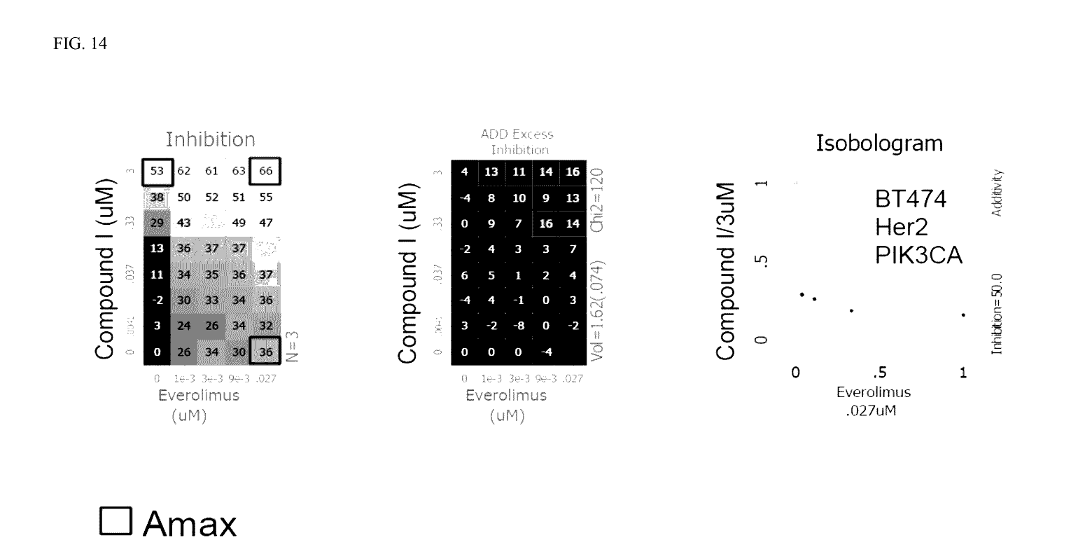

FIG. 14 shows full dose matrix cell proliferation data from single agent and concomitant everolimus (RAD001) and/or Compound I treatment in BT-474 human breast cancer cell models.

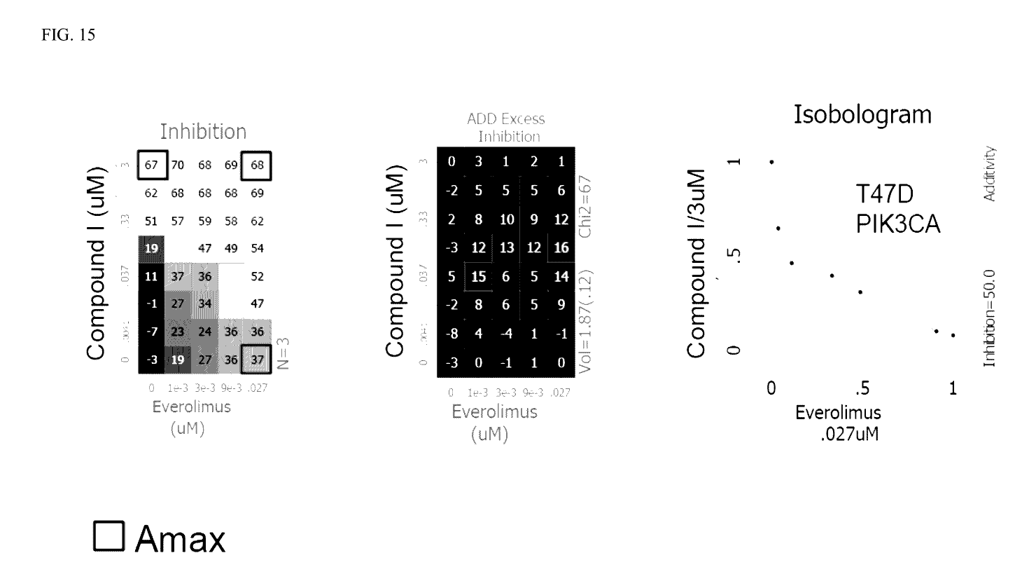

FIG. 15 shows full dose matrix cell proliferation data from single agent and concomitant everolimus (RAD001) and/or Compound I treatment in T47-D human breast cancer cell models.

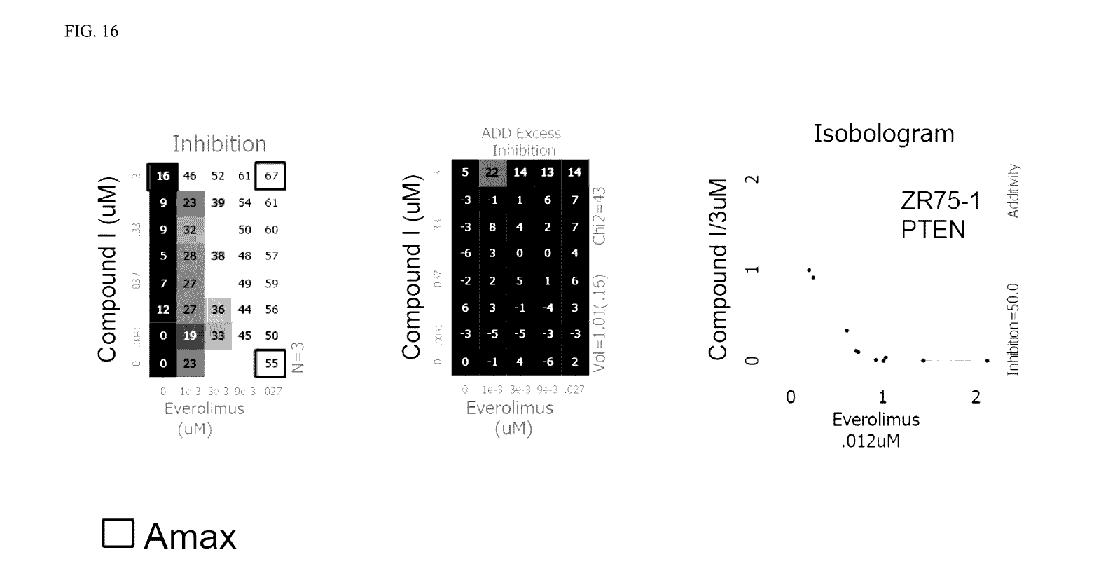

FIG. 16 shows full dose matrix cell proliferation data from single agent and concomitant everolimus (RAD001) and/or Compound I treatment in ZR-75-1 human breast cancer cell models.

### DETAILED DESCRIPTION OF THE INVENTION

The present invention relates to a pharmaceutical combination comprising (a) a compound of formula (A), as defined herein, or a pharmaceutically acceptable salt thereof, and (b) at least one mTOR inhibitor or a pharmaceutically acceptable salt thereof.

The following general definitions shall apply in this specification, unless otherwise specified:

The terms “comprising” and “including” are used herein in their open-ended and non-limiting sense unless otherwise noted.

The terms “a” and “an” and “the” and similar references in the context of describing the invention (especially in the context of the following claims) are to be construed to cover bot the singular and the plural, unless otherwise indicated herein or clearly contradicted by context. Where the plural form is used for compounds, salts, and the like, this is taken to mean also a single compound, salt, or the like.

“Combination” refers to either a fixed combination in one dosage unit form, or a kit of parts for the combined administration where a compound of the formula (A) and a combination partner (e.g. another drug as explained below, also referred to as “combination partner” or “therapeutic agent”) may be administered independently at the same time or separately within time intervals, especially where these time intervals allow that the combination partners show a cooperative, e.g. synergistic effect.

“Pharmaceutical combination” as used herein means a product that results from the mixing or combining of more than one active ingredient and includes both fixed and non-fixed combinations of the active ingredients. The term “fixed combination” or “fixed dose” means that the active ingredients, e.g. a compound of formula (A) and a combination partner, are both administered to a patient simultaneously in the form of a single entity or dosage. The term “non-fixed combination” means that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the warm-blooded animal in need thereof. The latter also applies to cocktail therapy, e.g. the administration of three or more active ingredients.

The term “a phosphatidylinositol 3-kinase inhibitor” is defined herein to refer to a compound which targets, decreases or inhibits PI 3-kinase. PI 3-kinase activity has been shown to increase in response to a number of hormonal and growth factor stimuli, including insulin, platelet-derived growth factor, insulin-like growth factor, epidermal growth factor, colony-stimulating factor, and hepatocyte growth factor, and has been implicated in processes related to cellular growth and transformation.

The term “pharmaceutical composition” is defined herein to refer to a mixture or solution containing at least one active ingredient or therapeutic agent to be administered to a warm-blooded animal, e.g., a mammal or human, in order to prevent or treat a particular disease or condition affecting the warm-blooded animal.

The term “pharmaceutically acceptable” is defined herein to refer to those compounds, materials, compositions and/or dosage forms, which are, within the scope of sound medical judgment, suitable for contact with the tissues a warm-blooded animal, e.g., a mammal or human, without excessive toxicity, irritation allergic response and other problem complications commensurate with a reasonable benefit/risk ratio.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the warm-blooded animal in need thereof. Alternatively, a therapeutically effective amount is sufficient to cause an improvement in a clinically significant condition/symptom in the warm-blooded animal in need thereof.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject.

The term “prevent”, “preventing” or “prevention” as used herein comprises the prevention of at least one symptom associated with or caused by the state, disease or disorder being prevented.

The term “jointly therapeutically active” or “joint therapeutic effect” as used herein means that the therapeutic agents may be given separately (in a chronologically staggered manner, especially a sequence-specific manner) in such time intervals that they prefer, in the warm-blooded animal, especially human, to be treated, still show a (preferably synergistic) interaction (joint therapeutic effect). Whether this is the case can, inter alia, be determined by following the blood levels, showing that both compounds are present in the blood of the human to be treated at least during certain time intervals.

WO2010/029082 describes specific 2-carboxamide cycloamino urea derivatives, which have been found to have inhibitory activity for PI3-kinases (phosphatidylinositol 3-kinases). These specific phosphatidylinositol 3-kinase (PI3K) inhibitors have advantageous pharmacological properties and show an improved selectivity for the PI3-kinase alpha as compared to the beta and/or delta and/or gamma subtypes. Specific 2-carboxamide cycloamino urea derivatives which are suitable for the present invention, their preparation and suitable formulations containing the same are described in WO2010/029082 and include compounds of formula (A)

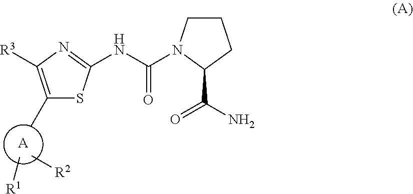

or a pharmaceutically acceptable salt thereof, wherein
A represents a heteroaryl selected from the group consisting of:

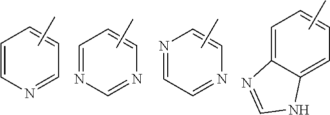

R¹ represents one of the following substituents: (1) unsubstituted or substituted, preferably substituted C₁-C₇-alkyl, wherein said substituents are independently selected from one or more, preferably one to nine of the following moieties: deuterium, fluoro, or one to two of the following moieties C₃-C₅-cycloalkyl; (2) optionally substituted C₃-C₅-cycloalkyl wherein said substituents are independently selected from one or more, preferably one to four of the following moieties: deuterium, C₁-C₄-alkyl (preferably methyl), fluoro, cyano, aminocarbonyl; (3) optionally substituted phenyl wherein said substituents are independently selected from one or more, preferably one to two of the following moieties: deuterium, halo, cyano, C₁-C₇-alkyl, C₁-C₇-alkylamino, di(C₁-C₇-alkyl)amino, C₁-C₇-alkylaminocarbonyl, di(C₁-C₇-alkyl)aminocarbonyl, alkoxy; (4) optionally mono- or di-substituted amine; wherein said substituents are independently selected from the following moieties: deuterium, C₁-C₇-alkyl (which is unsubstituted or substituted by one or more substituents selected from the group of deuterium, fluoro, chloro, hydroxy), phenylsulfonyl (which is unsubstituted or substituted by one or more, preferably one, C₁-C₇-alkyl, C₁-C₇-alkoxy, di(C₁-C₇-alkyl)amino-C₁-C₇-alkoxy); (5) substituted sulfonyl; wherein said substituent is selected from the following moieties: C₁-C₇-alkyl (which is unsubstituted or substituted by one or more substituents selected from the group of deuterium, fluoro), pyrrolidino, (which is unsubstituted or substituted by one or more substituents selected from the group of deuterium, hydroxy, oxo; particularly one oxo); (6) fluoro, chloro;
R² represents hydrogen;
R³ represents (1) hydrogen, (2) fluoro, chloro, (3) optionally substituted methyl, wherein said substituents are independently selected from one or more, preferably one to three of the following moieties: deuterium, fluoro, chloro, dimethylamino;
with the exception of (S)-Pyrrolidine-1,2-dicarboxylic acid 2-amide 1-({5-[2-(tert-butyl)-pyrimidin-4-yl]-4-methyl-thiazol-2-yl}-amide).

The radicals and symbols as used in the definition of a compound of formula (A) have the meanings as disclosed in WO2010/029082 which publication is hereby incorporated into the present application by reference in its entirety.

A preferred compound of the present invention is a compound which is specifically described in WO2010/029082. A very preferred compound of the present invention is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound I) or a pharmaceutically acceptable salt thereof. The synthesis of (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) is described in WO2010/029082 as Example 15.

Pharmaceutical combinations of the present invention include at least one compound which targets, decreases or inhibits the activity/function of serine/theronine mTOR kinase. Such compounds will be referred to an “mTOR inhibitor” and includes, but is not limited to, compounds, proteins or antibodies which target/inhibit the activity/function of members of the mTOR kinase family, e.g., RAD rapamycin (sirolimus which is also known by the name RAPAMUNE) and derivatives/analogs thereof such as everolimus (RAD001, Novartis) or compounds that inhibit the kinase activity of mTOR by directly binding to the ATP-binding cleft of the enzyme. Everolimus (RAID001) is also known by the name CERTICAN or AFINITOR.

Suitable mTOR inhibitors include e.g.:

I. Rapamycin which is an immunosuppressive lactam macrolide that is produced by Streptomyces hygroscopicus.

II. Rapamycin derivatives such as:
a. substituted rapamycin e.g. a 40-O-substituted rapamycin e.g. as described in U.S. Pat. No. 5,258,389, WO 94/09010, WO 92/05179, U.S. Pat. No. 5,118,677, U.S. Pat. No. 5,118,678, U.S. Pat. No. 5,100,883, U.S. Pat. No. 5,151,413, U.S. Pat. No. 5,120,842, WO 93/11130, WO 94/02136, WO 94/02485 and WO 95/14023 all of which are incorporated herein by reference;
b. a 16-O-substituted rapamycin e.g. as disclosed in WO 94/02136, WO 95/16691 and WO 96/41807, the contents of which are incorporated herein by reference;
c. a 32-hydrogenated rapamycin e.g. as described in WO 96/41807 and U.S. Pat. No. 5,256,790, incorporated herein by reference.
d. Preferred rapamycin derivatives are compounds of formula (B)

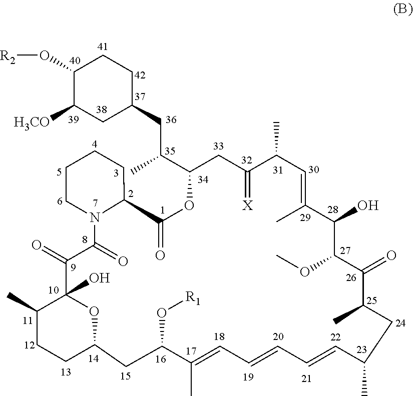

wherein
R₁ is CH₃ or C₃₋₆alkynyl,
R₂ is H or —CH₂—CH₂—OH, 3-hydroxy-2-(hydroxymethyl)-2-methyl-propanoyl or tetrazolyl, and X is ═O, (H,H) or (H₂OH)
provided that R₂ is other than H when X is ═O and R₁ is CH₃,
or a prodrug thereof when R₂ is —CH₂—CH₂—OH, e.g. a physiologically hydrolysable ether thereof.

Compounds of formula (B) are disclosed e.g. in International PCT Applications WO94/09010, WO95/16691 or WO 96/41807, which are incorporated herein by reference. They may be prepared as disclosed or by analogy to the procedures described in these references.

Preferred compounds are 32-deoxorapamycin, 16-pent-2-ynyloxy-32-deoxorapamycin, 16-pent-2-ynyloxy-32(S)-dihydro-rapamycin, 16-pent-2-ynyloxy-32(S)-dihydro-40-O-(2-hydroxyethyl)-rapamycin and, more preferably, 40-0-(2-hydroxyethyl)-rapamycin, disclosed as Example 8 in International PCT Application WO94/09010.

Particularly preferred rapamycin derivatives of formula (B) are 40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 32-deoxorapamycin, 16-pent-2-ynyloxy-32(S)-dihydro rapamycin, or TAFA-93.

e. Rapamycin derivatives also include so-called rapalogs, e.g. as disclosed in International PCT Applications WO98/02441 and WO01/14387, e.g. AP23573, AP23464, or AP23841.

Rapamycin and derivatives thereof have, on the basis of observed activity, e.g. binding to macrophilin-12 (also known as FK-506 binding protein or FKBP-12), e.g. as described in International PCT Applications WO94/09010, WO95/16691 or WO96/41807, been found to be useful e.g. as immunosuppressant, e.g. in the treatment of acute allograft rejection.

III. Ascomycin, which is an ethyl analog of FK506.

IV. AZD08055 (AstraZeneca) and OSI-027 (OSI Pharmaceuticals), which are compounds that inhibit the kinase activity of mTOR by directly binding to the ATP-binding cleft of the enzyme.

V. SAR543, deferolimus (AP23573/MK-8669, Ariad/Merck & Co.), AP23841 (Ariad), KU-0063794 (AstraZeneca/Kudos), INK-128 (Intellikine), EX2044, EX3855, EX7518, WYE-125132 (Wyeth), XL765 (Exelisis), NV-128 (Novogen), WYE-125132 (Wyeth), EM101/LY303511 (Emiliem).

A preferred mTOR inhibitor for the present invention is everolimus (RAD001). Everolimus (RAD001) has the chemical name ((1R,9S,12S,15R,16E,18R,19R, 21R,23S,24E,26E,28E,30S,32S,35R)-1,18-dihydroxy-12-{(1R)-2-[(1S,3R,4R)-4-(2-hydroxyethoxy)-3-methoxycyclohexyl]-1-methylethyl}-19,30-dimethoxy-15,17,21,23,29,35-hexamethyl-11,36-dioxa-4-aza-tricyclo[30.3.1.04,9] hexatriaconta-16,24, 26,28-tetraene-2,3,10,14,20-pentaone.) Everolimus and analogues are described in U.S. Pat. No. 5,665,772, at column 1, line 39 to column 3, line 11.

The structure of the active agents identified by code nos., generic or trade names may be taken from the actual edition of the standard compendium “The Merck Index” or from databases, e.g., Patents International (e.g., IMS World Publications). The corresponding content thereof is hereby incorporated by reference.

Comprised are likewise the pharmaceutically acceptable salts thereof, the corresponding racemates, diastereoisomers, enantiomers, tautomers, as well as the corresponding crystal modifications of above disclosed compounds (i.e., compounds of formula (A) and mTOR inhibitors) where present, e.g. solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

In one embodiment, the present invention provides a pharmaceutical combination comprising a compound of formula (A), or (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) specifically, or a pharmaceutically acceptable salt thereof and at least one mTOR inhibitor or a pharmaceutically acceptable salt thereof.

In one embodiment, the present invention provides invention provides a pharmaceutical combination comprising a compound of formula (A), or (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) specifically, or a pharmaceutically acceptable salt thereof and the mTOR inhibitor everolimus (RAD001) or a pharmaceutically acceptable salt thereof.

The pharmaceutical combinations of the present invention are useful in treating or preventing an mTOR kinase dependent disease in a warm-blooded animal in need thereof. Thus, in one aspect, the present invention provides a pharmaceutical combination comprising a compound of formula (A), or (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) specifically, or a pharmaceutically acceptable salt thereof and at least one mTOR inhibitor or a pharmaceutically acceptable salt thereof for use in treating or preventing an mTOR kinase dependent disease.

The term “mTOR kinase dependent diseases” includes but is not restricted to the following symptoms:
Organ or tissue transplant rejection, e.g. for the treatment of recipients of e.g. heart, lung, combined heart-lung, liver, kidney, pancreatic, skin or comeal transplants; graft-versus-host disease, such as following bone marrow transplantation;
Restenosis
Hamartoma syndromes, such as tuberous sclerosis or Cowden Disease
Lymphangioleiomyomatosis
Retinitis pigmentosis
Autoimmune diseases including encephalomyelitis, insulin-dependent diabetes mellitus, lupus, dermatomyositis, arthritis and rheumatic diseases
Steroid-resistant acute Lymphoblastic Leukaemia
Fibrotic diseases including scleroderma, pulmonary fibrosis, renal fibrosis, cystic fibrosis
Pulmonary hypertension
Immunomodulation
Multiple sclerosis
VHL syndrome
Carney complex
Familial adenonamtous polyposis
Juvenile polyposis syndrome
Birt-Hogg-Duke syndrome
Familial hypertrophic cardiomyopathy
Wolf-Parkinson-White syndrome
Neurodegenerative disorders such as Parkinson's, Huntington's, Alzheimer's and dementias caused by tau mutations, spinocerebellar ataxia type 3, motor neuron disease caused by SOD1 mutations, neuronal ceroid lipofucinoses/Batten disease (pediatric neurodegeneration)
wet and dry macular degeneration
muscle wasting (atrophy, cachexia) and myopathies such as Danon's disease.
bacterial and viral infections including M. tuberculosis, group A streptococcus, HSV type I, HIV infection
Neurofibromatosis including Neurofibromatosis type 1,
Peutz-Jeghers syndrome

Furthermore, “mTOR kinase dependent diseases” include proliferative diseases such as cancers and other related malignancies. A non-limiting list of the cancers associated with pathological mTOR signaling cascades includes breast cancer, renal cell carcinoma, gastric tumors, neuroendocrine tumors, lymphomas and prostate cancer.

Examples for a proliferative disease are for instance benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, lymphomas, a mammary carcinoma or a leukemia.

In a further aspect, the present invention provides the use of a compound of formula (A), or (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound I) specifically, or a pharmaceutically acceptable salt thereof and at least one mTOR inhibitor or a pharmaceutically acceptable salt thereof for the manufacture of a medicament for the treatment or prevention of an mTOR kinase dependent disease.

In another aspect the present invention provides a method of treating or preventing an mTOR kinase dependent disease by administering a compound of formula (A), or (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound I) specifically, or a pharmaceutically acceptable salt thereof and at least one mTOR inhibitor or a pharmaceutically acceptable salt thereof.

In another aspect the present invention provides a combination of a compound of formula (A), or (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound I) specifically, and at least one mTOR inhibitor selected from the group consisting of RAD rapamycin (sirolimus) and derivatives/analogs thereof such as everolimus (RAD001), temsirolimus (CCI-779), zotarolimus (ABT578), SAR543, ascomycin (an ethyl analog of FK506), deferolimus (AP235731 MK-8669), AP23841, KU-0063794, INK-128, EX2044, EX3855, EX7518, AZD08055, OSI-027, WYE-125132, XL765, NV-128, WYE-125132, and EM101/LY303511, wherein the active ingredients are present in each case in free form or in the form of a pharmaceutically acceptable salt, and optionally at least one pharmaceutically acceptable carrier, for simultaneous, separate or sequential use for the treatment of mammalian target of rapamycin (mTOR) kinase dependent diseases.

The present invention provides a method to reduce or block the phosphorylation and activation of AKT by mTOR inhibitors comprising administering a compound of formula (A) or a pharmaceutically acceptable salt thereof to a warm-blooded animal in need thereof. In another embodiment, the present invention provides a method of treating a proliferative disease dependent on acquired phosphorylation and activation of AKT during treatment with at least one mTOR inhibitor or a pharmaceutically acceptable salt thereof comprising administering a therapeutically effective amount of a compound of formula (A) or a pharmaceutically acceptable salt thereof to a warm-blooded animal in need thereof.

In another embodiment, the present invention relates to a method of treating a proliferative disease which has become resistant or has a decreased sensitivity to the treatment with at least one mTOR inhibitor or a pharmaceutically acceptable salt thereof comprising administering a therapeutically effective amount of a compound of formula (A) or a pharmaceutically acceptable salt thereof to a warm-blooded animal in need thereof. The resistance is e.g. due to phosphorylation and activation of AKT.

In a further aspect the present invention provides a method for improving efficacy of the treatment of a proliferative disease with at least one mTOR inhibitor or a pharmaceutically acceptable salt thereof comprising administering a combination comprising a compound of formula (A), or (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound I) specifically, or a pharmaceutically acceptable salt thereof and at least one mTOR inhibitor or a pharmaceutically acceptable salt thereof to a warm-blooded animal in need thereof.

The mTOR inhibitor used according to the present invention may be selected from RAD rapamycin (sirolimus) and derivatives/analogs thereof such as everolimus (RAD001), temsirolimus (CCI-779), zotarolimus (ABT578), SAR543, ascomycin (an ethyl analog of FK506), deferolimus (AP23573/MK-8669), AP23841, KU-0063794, INK-128, EX2044, EX3855, EX7518, AZD08055, O81-027, WYE-125132, XL765, NV-128, WYE-125132, and EM101/LY303511. Particularly preferred mTOR inhibitors in accordance with the present invention are sirolimus and/or everolimus.

The pharmaceutical compositions or combination in accordance with the present invention can be tested in clinical studies. Suitable clinical studies may be, for example, open label, dose escalation studies in patients with proliferative diseases. Such studies prove in particular the synergism of the active ingredients of the combination of the invention. The beneficial effects on proliferative diseases may be determined directly through the results of these studies which are known as such to a person skilled in the art. Such studies may be, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) may be administered in a fixed dose and the dose of agent (b) may be escalated. Each patient may receive doses of the agent (a) either daily or intermittent. The efficacy of the treatment may be determined in such studies, e.g., after 12, 18 or 24 weeks by evaluation of symptom scores every 6 weeks.

The administration of a pharmaceutical combination of the invention may result not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically active ingredients used in the combination of the invention.

A further benefit may be that lower doses of the active ingredients of the combination of the invention may be used, for example, that the dosages need not only often be smaller but may also be applied less frequently, which may diminish the incidence or severity of side-effects. This is in accordance with the desires and requirements of the patients to be treated.

It is one objective of this invention to provide a pharmaceutical composition comprising a quantity, which is jointly therapeutically effective at targeting or preventing a mammalian target of rapamycin (mTOR) dependent disease in a warm-blooded animal thereof, of (a) the compound of formula (A) or a pharmaceutically acceptable salt thereof and (b) at least one mTOR inhibitor or a pharmaceutically acceptable salt thereof, and optionally at least one pharmaceutically acceptable carrier. In this composition, the combination partners (a) and (b) can be administered together, one after the other or separately in one combined unit dosage form or in two separate unit dosage forms. The unit dosage form may also be a fixed combination.

In one aspect, the present invention provides a pharmaceutical composition comprising (a) a compound of formula (A) or a pharmaceutically acceptable salt thereof and (b) at least one mTOR inhibitor or pharmaceutically acceptable salt thereof, and optionally at least one pharmaceutically acceptable carrier. In one embodiment, the pharmaceutical composition comprises a quantity of the compound of formula (A) and at least one mTOR inhibitor which is jointly therapeutically effective against a mammalian target of rapamycin (mTOR) dependent disease.

In another aspect, the present invention provides a pharmaceutical combination comprising (a) a compound of formula (A) or a pharmaceutically acceptable salt thereof and (b) at least one mTOR inhibitor or pharmaceutically acceptable salt thereof, and optionally at least one pharmaceutically acceptable carrier, for simultaneous, separate or sequential use. Combination partners (a) and (b) may be administered together or separately to a warm-blooded animal in need thereof.

In accordance with the present invention, the pharmaceutical compositions for the separate administration of combination partner (a) and combination partner (b) or for the administration in a fixed combination, i.e. a single galenical composition comprising at least two combination partners (a) and (b) may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers or diluents, especially suitable for enteral or parenteral application.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

Suitable pharmaceutical compositions may contain, for example, from about 0.1% to about 99.9%, preferably from about 1% to about 60%, of the active ingredient(s). The actual amount of the compound of formula (A) and the mTOR inhibitor administered in accordance with the present invention will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

The compound of formula (A) may be administered in therapeutically effective amounts ranging from about 0.05 to about 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-700 mg per day.

The mTOR inhibitor everolimus (RAD001) may be administered to a human in a daily dosage range of 0.5 to 1000 mg; preferably in the range of 0.5 mg to 15 mg; most preferably in the range of 0.5 mg to 10 mg.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise (i) administration of the first agent (a) in free or pharmaceutically acceptable salt form and (ii) administration of an agent (b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

The present invention further comprises the following embodiments:
A synergistic combination for human administration comprising a compound of formula (A) which is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}amide) and at least one mTOR inhibitor, in free form or in the form of a salt thereof, in a combination range which corresponds to a synergistic combination range of approximately 330 nM-3 μM and approximately 1 nM-27 nM respectively in the SKBR-3 breast cancer cell model or the BT-474 breast cancer cell model.
A synergistic combination for human administration comprising a compound of formula (A) which is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) and at least one mTOR inhibitor, in free form or in the form of a salt thereof, in a combination range which corresponds to a synergistic combination range of approximately 12 nM-100 nM and approximately 1 nM-27 nM respectively in the T47-D breast cancer cell model.
A synergistic combination for human administration comprising a compound of formula (A) which is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-}-amide) and at least one mTOR inhibitor, in free form or in the form of a salt thereof, in a combination range which corresponds to a synergistic combination range of approximately 3 μM and approximately 1 nM 27 nM respectively in the ZR-75-1 breast cancer cell model.

The following examples are illustrative only and not intended to be limiting.

### Example 1: Effect of the Combination of Everolimus (RAD001) with Compound I in BT474 and MDA-MB-231 Breast Tumor Cells Detected by Western Blot Analysis

### Material and Methods

Preparation of Compounds:

The compound everolimus (RAD001) is synthesized by Novartis Pharma AG. A 20 mM stock solution is prepared in DMSO and stored −20° C. A 10 mM stock solution of the (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) is prepared in DMSO and stored at −20° C.

Cells and Cell Culture Conditions:

Human breast carcinoma BT474 cells (ATCC HTB-26) and MDA-MB-231 (ATCC HTB-20) are obtained from the American Type Culture Collection (ATCC, Rockville, Md., USA).

BT474 cells are maintained in Hybri-Care medium (ATCC) supplemented with 10% v/v fetal calf serum and 2 mM L-glutamine. MDA-MB-231 cells are grown in RPMI 1640 medium (Amimed, Allschwil, Switzerland) supplemented with 10% v/v fetal calf serum and 2 mM L-glutamine. All media are supplemented with 100 μg/mL penicillin/streptomycin and cells are maintained at 37° C. in 5% CO2.

Cell Treatment and Cell Extraction:

BT474 and MDA-MB-231 cells are seeded at a density of 3.3×10⁴ cells/cm² and 1.6×10⁴ cells/cm², respectively, and incubated for 48 h at 37° C. and 5% CO₂, prior to treatment with DMSO vehicle, 20 nM RAD001 and/or various concentrations of Compound I for 24 h.

Cell lysates are prepared as follows. Culture plates are washed once with ice-cold PBS containing 1 mM PMSF and once with ice-cold extraction buffer [50 mM Hepes (pH 7.4), 150 mM NaCl, 25 mM 6-glycerophosphate, 25 mM NaF, 5 mM EGTA, 1 mM EDTA, 15 mM PPi, 2 mM sodium orthovanadate, 10 mM sodium molybdate, leupeptin (10 μg/mL), aprotinin (10 μg/mL), 1 mM DTT and 1 mM PMSF]. Protease inhibitors are purchased from SIGMA Chemical, St. Louis, Mo. Cells are extracted in the same buffer, containing 1% NP-40 (SIGMA Chemicals). The extracts are homogenized, cleared by centrifugation, aliquoted and frozen at −80° C. Protein concentration is determined with the BCA Protein Assay (Pierce, Rockford, Ill., USA).

Immunoblotting:

Twenty micrograms of cell extracts are resolved electrophoretically on 12% denaturing sodium dodecyl sulfate polyacrylamide gels (SOS-PAGE) and are transferred to polyvinylidene difluoride filters (PVDF; Millipore Corporation, Bedford, Mass., USA) by wet-blotting (1 h at 250 mA) and are probed overnight at 4° C. with the following primary antibodies:
(a) anti-phospho-Akt (S₄₇₃) (clone 14-05; 1:2000) is obtained from DAKO (Glostrup, Denmark) and diluted in PBS, 0.5% v/v Tween, 0.5% w/v milk.
(b) anti-phospho-MAPK (T₂₀₂/Y₂₀₄) (clone ECA297; 1:50) is obtained from DAKO (Glostrup, Denmark) and diluted in PBS, 0.5% v/v Tween, 0.5% w/v milk.
(c) anti-phospho-MEK 1/2 (S₂₁₇/S₂₂₁) (cat #9154; 1:1000) is obtained from Cell Signaling Technology and diluted in PBS, 0.1% v/v Tween, 0.5% w/v milk.
(d) anti-Actin (cat # MAB1501; 1:20,000) is obtained from Chemicon (Billerica, Mass., USA) and diluted in PBS, 0.1% v/v Tween.

After incubation with the appropriate primary antibody (as listed above), decorated proteins are revealed using horseradish peroxidase-conjugated anti-mouse or anti-rabbit immunoglobulins followed by enhanced chemiluminescence (ECL Plus kit; Amersham Pharmacia Biotech, Buckinghamshire, UK) and are quantified using Quantity One Software (Bio-Rad, Munich, Germany).

Each cell extract is further quantified by Reverse protein Array methodology as described as follows.

Each cell extracts are spotted onto ZeptoMARK® PWG protein microarray chips (Zeptosens, Witterswil, Switzerland) with the piezoelectric microdispense-based, non-contact Nano-Plotter 2.1 (GeSiM, Grosserkmannsdorf, Germany). After spotting the ZeptoMARK® protein microarrays, the chips are incubated for 1 hour at 37° C. To receive a uniform blocking result, the CeLyA blocking buffer BB1 (Zeptosens, cat. No. 9040) is administered via an ultrasonic nebulizer. After 30 minutes of blocking the chips are extensively rinsed with deionized water (Milli-Q quality, 18MΩ×cm) and dried in a nitrogen airflow.

After the sample spotting and blocking procedure, the ZeptoMARK® chips are transferred to the ZeptoCARRIER (Zeptosens, cat. No. 1100), whose six flow cells individually address the six arrays on a chip, and are washed twice with 200 μl CAB1 CeLyA assay buffer (Zeptosens, cat. No. 9032). The assay buffer is then aspirated and each compartment is incubated with 100 μl of the primary target antibody (pAkt Ser473: CST#4060; pAkt Thr308: CST#2965, Aktl pan: Epitomics #1085-1) at RT over night. After incubation, the primary antibody is removed, the arrays are washed twice with CAB1 buffer and are further incubated with 100 μl of Alexa fluor 647-labeled anti rabbit IgG Fib fragments (Nitrogen; #Z25305) for one hour at RT in the dark. After incubation, the arrays were washed twice with 200 μl CAB1 buffer. The fluorescence of the target-bound Fib fragments is read out on the ZeptoReader (Zeptosens, Witterswil, Switzerland) using a laser (excitation wavelength 635 nm) and a CCD camera. The fluorescence signal was assessed with exposure times of 1, 3, 5 and 10 seconds, depending on the intensity of the signal.

The fluorescence images for each array are analyzed with the ZeptoVIEW Pro 2.0 software (Zeptosens, Witterswil, Switzerland) and the RFI for each signal is calculated. Antibodies and antibody dilutions used in this experiment:

| Antigen | Provider | Ref | Dilution |
|---|---|---|---|
| pAkt Ser473 | Cell Signaling | 4060 | 1/500 |
| | Technology | | |
| Akt 1 pan | Epitomics | 1085-1 | 1/500 |
| Zenon ® Alexa | Invitrogen | Z25305 | 1/500 |
| Fluor 647 rabbit | | | |

### Results:

The phosphorylation levels of AKT(S473), MARK (T202/Y204), MEK1/2 (S217/S221) and total actin levels in the presence of everolimus (RAD001) and everolimus (RAD001) in combination with Compound I in BT474 breast tumor cells determined by Western blot analysis are depicted in FIG. 1.

The phosphorylation levels of AKT(S473), AKT (T308) and total AKT levels in the presence of everolimus (RAD001) and everolimus (RAD001) in combination with Compound I in BT474 breast tumor cells as quantified by Reverse protein Array are depicted in FIGS. 2 to 4 respectively.

The phosphorylation levels of AKT(S473), MAPK (T202/Y204) and total actin levels in the presence of everolimus (RAD001) and everolimus (RAD001) in combination with Compound I in MDA-MB231 breast tumor cells determined by Western blot analysis are depicted in FIG. 5.

The phosphorylation levels of AKT(S473), AKT (T308) and total AKT levels in the presence of everolimus (RAD001) and everolimus (RAD001) in combination with Compound I in MDA-MB231 breast tumor cells as quantified by Reverse protein Array are depicted in FIGS. 6 to 8 respectively.

The phosphorylation levels of AKT(S473) and total AKT levels in the presence of everolimus (RAD001) and everolimus (RAD001) in combination with Compound I in MDA-MB231 breast tumor cells determined by Western blot analysis and Quantified using the Quantity One Software as showed in FIG. 9, in a second set of experiment.

The phosphorylation levels of AKT(S473), AKT (T308) and total AKT levels in the presence of everolimus (RAD001) and everolimus (RAD001) in combination with Compound I in MDA-M8231 breast tumor cells as quantified, in a second set of experiment, by Reverse protein Array are depicted in FIGS. 10 to 12 respectively.

### Example 2: Effect of the Combination of Everolimus (RAD001) with Compound I in SKBR-3 Human Breast Cancer Cell Model

### Material and Methods

The human breast cancer cell line SKBR-3 is purchased from American Type Cell Collection. The SKBR-3 human breast cancer cell line is HER2 amplified. The SKBR-3 human breast cancer cell line is cultured at 37° C. in a 5% CO₂ incubator in RPMI 1640 (ATCC #30-2001) or other suggested media complemented with 10% fetal bovine serum, 2 mmol/L glutamine and 1% sodium pyruvate.

Cell Proliferation Assay:

Cell viability is determined by measuring cellular ATP content using the CellTiter-Glo® Luminescent Cell Viability Assay (Promega #G7573) according to manufacturer's protocol. Briefly, 1500-50000 cells are plated on either 384 or 96 well plates in 25 μl (384 well) or 100 μl (96 well) growth media, cells are allowed to attach overnight and followed by 72 hrs of incubation with various concentration of drugs or drug combinations, at the end of the drug treatment, equal volume of the CellTiter-Glo regent are added to each well to lyse the cell, and luminescence signals are recorded on a Envision plate reader.

Method for Calculating the Effect of the Combination:

To evaluate the everolimus (RAD001) and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) combination effect and to identify potential synergistic effect at all possible concentrations, the combination studies are conducted with a “dose matrix”, where a combination is tested in all possible permutations of serially-diluted everolimus (RAD001) and Compound I single agent doses, in all combination assays, compounds are applied simultaneously. Single agent dose responding curves, IC₅₀, IC₉₀, and the Synergy are all analyzed using Chalice software (CombinatoRx, Cambridge Mass.). Synergy is calculated by comparing a combination's response to those of its single agents, against the drug-with-itself dose-additive reference model. Deviations from dose additivity can be assessed visually on an lsobologram or numerically with a Combination Index. Excess inhibition compare to additivity can also be plotted as a full dose-matrix chart to capture where the synergies occur. To quantify the overall strength of combination effects, a volume score V_{HSA}=Σ_{X,Y} Inf_{X} Inf_{Y} (I_{data}−I_{HSA}) is also calculated between the data and the highest-single-agent surface, normalized for single agent dilution factors f_{X},f_{Y}.

### Results:

The effect of single agent and concomitant everolimus (RAD001)/Compound I treatment on cell proliferation is evaluated using the cell titer glow (CTG) assay described above. The cells are plated at 3000 cells per well in 384 well plates in triplicates, and treated with compound for 72 hrs before the measurement (FIG. 13). In this “dose matrix” study, everolimus (RAD001) is subjected to a 4 dose 3× serial dilution with the high dose at 27 nM and the low dose at 1 nM, and Compound I is subjected to a 7 dose 3× serial dilution with high dose at 3 μM and low dose at about 4 nM.

The results of this study are set forth in FIG. 13. Compound I alone causes a concentration-dependent inhibition of cell growth with the Aₘₐₓ, the maximum fraction of inhibition=0.40 (40% growth inhibition compare to DMSO control); everolimus (RAD001) displaces a similar level of minor growth inhibitory effect on cell proliferation as a single agent, never achieved an IC₅₀, and the Aₘₐₓ=0.32. Concomitant everolimus (RAD001)/Compound I treatment significantly boosts the maximum level of inhibition, with Aₘₐₓ=0.63 compared to either single agents (everolimus (RAD001)=0.32, and Compound I Aₘₐₓ=040). Over the entire dose matrix, enhanced synergistic activities are observed for everolimus (RAD001) at all doses (1 nM-27 nM) and part of the higher dose ranges for Compound I (330 nM-3 μM). At relatively low Compound I concentrations (4 nM-37 nM), the combination does not seem to exhibit additional benefit compared to Compound I and everolimus (RAD001) as single agent treatments in this experiment.

### Example 3: Effect of the Combination of Everolimus (RAD001) with Compound I in BT-474 Breast Tumor Cells

### Material and Methods

The human breast cancer cell line BT-474 is purchased from American Type Cell Collection. The BT-474 human breast cancer cell line includes both PIK3CA mutation and HER2 amplification. The BT-474 breast cancer cell line is cultured at 37° C. in a 5% CO₂ incubator in RPMI 1640 (ATCC #30-2001) or other suggested media complemented with 10% fetal bovine serum, 2 mmol/L glutamine and 1% sodium pyruvate.

Cell Proliferation Assay:

Cell viability is determined by measuring cellular ATP content using the CellTiter-Glo® Luminescent Cell Viability Assay (Promega #G7573) according to manufacturers protocol. Briefly, 1500-50000 cells are plated on either 384 or 96 well plates in 25 μl (384 well) or 100 μl (96 well) growth media, cells are allowed to attach overnight and followed by 72 hrs of incubation with various concentration of drugs or drug combinations, at the end of the drug treatment, equal volume of the CellTiter-Glo regent are added to each well to lyse the cell, and luminescence signals are recorded on a Envision plate reader.

Method for Calculating the Effect of the Combination:

To evaluate the everolimus (RAD001) and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) combination effect and to identify potential synergistic effect at all possible concentrations, the combination studies are conducted with a “dose matrix”, where a combination is tested in all possible permutations of serially-diluted everolimus (RAD001) and Compound I single agent doses, in all combination assays, compounds are applied simultaneously. Single agent dose responding curves, IC₅₀, IC₉₀, and the Synergy are all analyzed using Chalice software (CombinatoRx, Cambridge Mass.). Synergy is calculated by comparing a combination's response to those of its single agents, against the drug-with-itself dose-additive reference model. Deviations from dose additivity can be assessed visually on an Isobologram or numerically with a Combination Index. Excess inhibition compare to additivity can also be plotted as a full dose-matrix chart to capture where the synergies occur. To quantify the overall strength of combination effects, a volume score V_{HSA}=Σ_{X,Y} Inf_{X} Inf_{Y} (I_{data}−I_{HSA}) is also calculated between the data and the highest-single-agent surface, normalized for single agent dilution factors f_{X},f_{Y}.

### Results:

The effect of single agent and concomitant everolimus (RAD001)/Compound I treatment on cell proliferation is evaluated using the cell titer glow (CTG) assay described above. The effect of single agent and concomitant everolimus (RAD001)/Compound I treatment on cell proliferation is evaluated using the cell titer glow (CTG) assay described above. The experiment setup is identical to the experiment procedure described above for the SKBR-3 model (FIG. 14). And the same “dose matrix” (everolimus (RAD001): 4 dose, 3×, 1 nM to 27 nM, Compound I: 7 dose, 3×, 4 nM to 3 μM) is applied.

The results of this study are set forth in FIG. 14. Compound I alone causes a concentration-dependent inhibition of cell growth with the IC₅₀ around 3 μM and Aₘₐₓ around 0.53 (53% growth inhibition compare to DMSO control); everolimus (RAD001) displaces a minor growth inhibitory effect on cell proliferation as a single agent, never achieves an IC₅₀, and Aₘₐₓ=0.36. Concomitant everolimus (RAD001)/Compound I treatment significantly boosts the maximum level of inhibition, with Aₘₐₓ=0.66 compared to either single agents (everolimus (RAD001) Aₘₐₓ=0.36, and Compound I Aₘₐₓ=0.53). Over the entire dose matrix, enhanced synergistic activities are observed for everolimus (RAD001) at all doses (1 nM-27 nM) and the high dose Compound I (330 nM-3 μM). At lower Compound I concentrations (4 nM-37 nM), the combination does not seem to exhibit additional benefit compared to Compound I and everolimus (RAD001) as single agent treatments in this experiment.

### Example 4: Effect of the Combination of Everolimus (RAD001) with Compound I in T47-D Human Breast Cancer Cell Model

### Material and Methods

The human breast cancer cell line T47-D is purchased from American Type Cell Collection. The T47-D human breast cancer cell line includes PIK3CA mutation. The T47-D human breast cancer cell line is cultured at 37° C. in a 5% CO₂ incubator in RPMI 1640 (ATCC #30-2001) or other suggested media complemented with 10% fetal bovine serum, 2 mmol/L glutamine and 1% sodium pyruvate.

Cell Proliferation Assay:

Cell viability is determined by measuring cellular ATP content using the CellTiter-Glo® Luminescent Cell Viability Assay (Promega #G7573) according to manufacturer's protocol. Briefly, 1500-50000 cells are plated on either 384 or 96 well plates in 25 μl (384 well) or 100 μl (96 well) growth media, cells are allowed to attach overnight and followed by 72 hrs of incubation with various concentration of drugs or drug combinations, at the end of the drug treatment, equal volume of the CellTiter-Glo regent are added to each well to lyse the cell, and luminescence signals are recorded on a Envision plate reader.

Method for Calculating the Effect of the Combination:

To evaluate the everolimus (RAD001) and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) combination effect and to identify potential synergistic effect at all possible concentrations, the combination studies are conducted with a “dose matrix”, where a combination is tested in all possible permutations of serially-diluted everolimus (RAD001) and Compound I single agent doses, in all combination assays, compounds are applied simultaneously. Single agent dose responding curves, IC₅₀, IC₉₀, and the Synergy are all analyzed using Chalice software (CombinatoRx, Cambridge Mass.). Synergy is calculated by comparing a combination's response to those of its single agents, against the drug-with-itself dose-additive reference model. Deviations from dose additivity can be assessed visually on an Isobologram or numerically with a Combination Index. Excess inhibition compare to additivity can also be plotted as a full dose-matrix chart to capture where the synergies occur. To quantify the overall strength of combination effects, a volume score V_{HSA}⁼Σ_{X,Y} Inf_{X} Inf_{Y} (I_{data}−I_{HSA}) is also calculated between the data and the highest-single-agent surface, normalized for single agent dilution factors f_{X},f_{Y}.

### Results:

The effect of single agent and concomitant everolimus (RAD001)/Compound I treatment on cell proliferation is evaluated using the cell titer glow (CTG) assay described above. The effect of single agent and concomitant everolimus (RAD001)/Compound I treatment on cell proliferation is evaluated using the cell titer glow (CTG) assay described above. The experiment setup is identical to the experiment procedure described above for the SKBR-3 model (FIG. 15). And the same “dose matrix” (everolimus (RAD001): 4 dose, 3×, 1 nM to 27 nM, Compound I: 7 dose, 3×, 4 nM to 3 μM) is applied.

The results of this study are set forth in FIG. 15. Compound I alone causes a significant concentration-dependent inhibition of cell growth with the IC₅₀ around 330 nM and Aₘₐₓ around 0.67 (67% growth inhibition compare to DMSO control); everolimus (RAD001) displaces a minor growth inhibitory effect on cell proliferation as a single agent, never achieved an IC₅₀, and Aₘₐₓ=0.37. Concomitant everolimus (RAD001)/Compound i treatment does not boost the maximum level of inhibition, with Aₘₐₓ=0.68, comparable to the single agent Compound I treatment (Aₘₐₓ=0.67). Over the entire dose matrix, slightly enhanced and weakly synergistic activities are observed for everolimus (RAD001) at all doses (1 nM-27 nM) and the relatively dose Compound I (12 nM-100 nM). At both high and low end of Compound I concentrations (4 nM, 330 nM-3 μM), the combination does not seem to exhibit additional benefit compare to Compound I and everolimus (RAD001) as single agent treatments in this experiment.

### Example 5: Effect of the Combination of Everolimus (RAD001) with Compound I in ZR-75-1 Human Breast Cancer Cell Model

### Material and Methods

The human breast cancer cell line ZR-75-1 is purchased from American Type Cell Collection. The ZR-75-1 human breast cancer cell line includes PTEN mutation. The ZR-75-1 human breast cancer cell line is cultured at 37° C. in a 5% CO₂ incubator in RPMI 1640 (ATCC #30-2001) or other suggested media complemented with 10% fetal bovine serum, 2 mmol/L glutamine and 1% sodium pyruvate.

Cell Proliferation Assay:

Cell viability is determined by measuring cellular ATP content using the CellTiter-Glo® Luminescent Cell Viability Assay (Promega #G7573) according to manufacturers protocol. Briefly, 1500-50000 cells are plated on either 384 or 96 well plates in 25 μl (384 well) or 100 μl (96 well) growth media, cells are allowed to attach overnight and followed by 72 hrs of incubation with various concentration of drugs or drug combinations, at the end of the drug treatment, equal volume of the CellTiter-Glo regent are added to each well to lyse the cell, and luminescence signals are recorded on a Envision plate reader.

Method for Calculating the Effect of the Combination:

To evaluate the everolimus (RAD001) and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) combination effect and to identify potential synergistic effect at all possible concentrations, the combination studies are conducted with a “dose matrix”, where a combination is tested in all possible permutations of serially-diluted everolimus (RAD001) and Compound I single agent doses, in all combination assays, compounds are applied simultaneously. Single agent dose responding curves, IC₅₀, IC₉₀, and the Synergy are all analyzed using Chalice software (CombinatoRx, Cambridge Mass.). Synergy is calculated by comparing a combination's response to those of its single agents, against the drug-with-itself dose-additive reference model. Deviations from dose additivity can be assessed visually on an Isobologram or numerically with a Combination Index. Excess inhibition compare to additivity can also be plotted as a full dose-matrix chart to capture where the synergies occur. To quantify the overall strength of combination effects, a volume score V_{HSA}=Σ_{X,Y} Inf_{X} Inf_{Y} (I_{data}−I_{HSA}) is also calculated between the data and the highest-single-agent surface, normalized for single agent dilution factors f_{X},f_{Y}.

### Results:

The effect of single agent and concomitant everolimus (RAD001)/Compound I treatment on cell proliferation is evaluated using the cell titer glow (CTG) assay described above. The effect of single agent and concomitant everolimus (RAD001)/Compound I treatment on cell proliferation is evaluated using the cell titer glow (CTG) assay described above. The experiment setup is identical to the experiment procedure described above for the SKBR-3 model (FIG. 16). And the same “dose matrix” (everolimus (RAD001): 4 dose, 3×, 1 nM to 27 nM, Compound I: 7 dose, 3×, 4 nM to 3 μM) is applied.

The results of this study are set forth in FIG. 16. Compound I alone does not caused significant inhibition on cell growth with Aₘₐₓ around 0.16 (16% growth inhibition compare to DMSO control); everolimus (RAD001) displaces better growth inhibitory effect on cell proliferation as a single agent, with IC₅₀ around 15 nM and Aₘₐₓ=0.55. Concomitant everolimus (RAD001)/Compound I treatment significantly boosts the maximum level of inhibition, with Aₘₐₓ=0.67 compare to either single agents (everolimus (RAD001) Aₘₐₓ=0.55, and Compound I Aₘₐₓ=0.16). However, over the entire dose matrix, significant and weakly synergistic effect boost is observed at the highest dose of Compound 1 (3 μM). At lower dose range of Compound I (4 nM-1 μM), the combination does not seem to exhibit additional benefit compared to Compound I and everolimus (RAD001) as single agent treatments in this experiment.

### Example 6: Effect of the Combination of Everolimus (RAD001) with Compound I in DU145 Human Prostate Carcinoma Nude Mouse Xenograft Model

### Methods and Materials

8 weeks old, male nude mice (nu/nu, Harlan) having a body weight (BW) range of 21.0-31.3 g on Day 1 of the study are used. The animals are fed ad libitum water (reverse osmosis, 1 ppm CI) and NIH 31 Modified and Irradiated lab Diet® consisting of 18.0% crude protein, 5.0% crude fat, and 5.0% crude fiber. The mice are housed on irradiated Enrich-o'Cobs™ Laboratory Animal Bedding in static microisolators on a 12-hour light cycle at 21-22° C. and 40-60% humidity.

The DU145 human prostate carcinoma cell line is obtained from the American Type Culture Collection (ATCC). The DU145 cell line is maintained as exponentially growing cultures in RPMI-1640 medium supplemented with 10% fetal bovine serum, 2 mM glutamine, 100 units/mL penicillin G sodium, 100 μg/mL streptomycin sulfate, 2 μg/mL gentamicin, 10 mM HEPES, and 0.075% sodium bicarbonate. The tumor cells are cultured in tissue culture flasks in a humidified incubator at 37° C., in an atmosphere of 5% CO₂ and 95% air.

DU145 prostate carcinoma cells are harvested during exponential growth and resuspended at a concentration of 5×10⁷ cells/mL in cold PBS with 50% Matrigel™ (BD Biosciences). Each nude mouse is inoculated subcutaneously in the right flank with 0.2 mL of the suspension (1×10⁷ cells). The tumors are calipered in two dimensions to monitor growth as their mean volume approached the desired 100-150 mm³ range. Tumor size, in mm³, is calculated from: Tumor Volume=(width²×length)/2. Tumor weight can be estimated with the assumption that 1 mg is equivalent to 1 mm³ of tumor volume. Seven days after tumor implantation, on Day 1 of the study, mice with individual tumor sizes of 144-196 mm³ are sorted into 11 groups of ten mice, with a group mean tumor volume of 181-184 mm³.

(S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound I”) is stirred in N-methylpyrrolidone (NMP; 10% of final volume) at room temperature until dissolved. Polyethylene glycol 300 (PEG300) is added (30% of final volume), followed by Solutol® HSS15 (20% of final volume), and the mixture is stirred until homogenous. The final volume is achieved by addition of deionized water (40% of final volume). The Compound I vehicle, NMP: PEG300: Solutol® HS15: deionized water (10:30:20:60), is designated as “Vehicle 1”. Solutions for the lower doses are prepared by dilution of the high-dose solution with Vehicle 1. Fresh dosing solutions are prepared weekly and stored at 4° C., protected from light.

Everolimus (RAD001) is formulated in a microemulsion that contained 2% (w/w) active ingredient, i.e. 20 mg RAD001/g; the density of the microemulsion is 0.995 g/cm³. The RAD001 microemulsion is aliquotted and initially stored at −20° C. An aliquot of the stock is thawed, divided into weighed daily portions, ad stored at 4° C. On each treatment day, a RAD001 aliquot is brought to room temperature and diluted with dextrose in water (D5W) to provide a 1 mg/mL solution for the highest dose. This stock is diluted with D5W to prepare solutions for the lower doses. The placebo microemulsion, diluted with D5W, is designated as “Vehicle 2”.

### Treatment Plan:

Compound I, RAD001, and their vehicles are each administered by oral gavage (p.o.) once daily for twenty-one consecutive days (qd×21). For combination threapies on Days 1-20, RAD001 is dosed within 30 minutes after Compound I. On Day 21 and on Day 7 in Group 10, RAD001 followed by Compound I immediately, on a cage by cage basis. Paclitaxel is administered via bolus tail veil injections (i.v.) once daily on alternate days for five doses (qod×5). The dosing volume, 10 mL/kg 0.2 mL/20 g mouse), is scaled to the weight to each animal as determined on the day of dosing, except on weekends, when the Friday BWs are carried forward.

11 groups of nude mice (n=10 per group) are treated as follows: Group 1 mice receives Vehicle 1 and Vehicle 2, and served as controls for all analyses. Groups 2-4 receives monotherapies with 12.5, 25, and 50 mg/kg Compound I, respectively. Groups 5-7 receives monotherapies with 2.5, 5, and 10 mg/kg RAD001, respectively. Group 8 receives 12.5 mg/kg Compound I in combination with 10 mg/kg RAD001. Group 9 receives 25 mg/kg Compound I in combination with 5 mg/kg RAD001. Group 10 receives 50 mg/kg Compound I in combination with 2.5 mg/kg RAD001; because of toxicity, this treatment is stopped after seven doses of each agent (qd×7). Group 11 receives 25 mg/kg paclitaxel.

### Tumor Growth Inhibition:

Treat efficacy is determined on Day 21. For the purpose of statistical and graphical analyses, ΔTV, the difference in tumor volume between Day 1 (the start of dosing) and the endpoint day, is determined for each animal that survives to Day 21. For each treatment group, the response on the endpoint day is calculated by the following relation:

T/C(%)=100×ΔT/ΔC, for ΔT>0

where, ΔT=(mean tumor volume of the treated group on the endpoint day)−(mean tumor volume of the treated group on Day 1), and ΔC=(mean tumor volume of the control group on the endpoint day)−(mean tumor volume of the control group on Day 1). A treatment that achieves a T/C value of 40% or less may be classified as potentially therapeutically active.

### Criteria for Regression Responses:

Treat efficacy may also be determined from the number of regression responses. Treatment may cause a partial regression (PR) or a complete regression (CR) of the tumor in an animal. A PR indicates that the tumor volume is 50% or less of its Day 1 volume for three consecutive measurements during the course of the study, and equal to or greater than 13.5 mm³ for one or more of these three measurements. A CR indicates that the tumor volume is less than 13.5 mm³ for three consecutive measurements during the course of the study.

### Toxicity:

Animals are weighed on Days 1-5, and on each treatment day (except weekend days) until the end of the study. Acceptable toxicity for the maximum tolerated dose is defined as a group mean BW loss of less than 15% during the test, and not more than one treatment-related (TR) death among ten animals. Any animal with BW losses exceeding 20% for one measurement, is to be euthanized and classified as a TR death, unless it is the first death in the group. Non-treatment-related (NTR) deaths are to be categorized as NTRa (due to accident or error), NTRu (due to unknown causes), or NTRm (necropsy-confirmed tumor dissemination by invasion and/or metastasis). To conserve animals while providing maximum information the first death in a group is to be classified as NTRu, but the death is to be reclassified as TR if subsequent group performance shows that the treatment is toxic.

### Sampling:

On Day 7, Animals #1-4 in Group 10 are euthanized 2 hours post-dosing by terminal cardiac puncture under CO₂ anesthesia. On Day 8, at 24 hours post-dosing, Animal #5 is sampled likewise. Full volume blood is collected from each animal and individually processed for plasma with K-EDTA as anticoagulant. The plasma samples are frozen at −80° C. Tumors are excised and snap frozen in liquid N2. At 2 and 24 hours post-final dosing on Day 21, four animals per time point in Groups 1, 4, 6 and 9 are sampled for blood and tumors as previously described.

In the raw data, Group 4 Animals #2, 6 and 7 exit the study as TR deaths; and Group 9 Animals #4 and 10 exit as TR and NTR, respectively. These animals actually survive to Day 21 and are sampled.

### Statistical and Graphical Analyses:

Statistical and graphical analyses are performed with Prism 3.03 (Graph Pad) for Windows. The significance of differences between the mean ΔTV values for treated versus control groups of mice is determined by analysis of variance (ANOVA), with Bartlett's test, and a post-hoc Dunnett's multiple comparison test. When Bartlett's test indicated significant differences among the variances (P<0.0001), the results are analyzed with the nonparametric Kruskal-Wallis test, which shows significant differences among the median volume changes (P<0.0001). Differences between groups are analyzed post-hoc with Dunn's multiple comparison test. The Mann-Whitney U test is employed to compare median volume changes in two groups. The two-tailed statistical analyses are conducted at P=0.05. Prism summarizes test results as not significant (ns) at P>0.05, significant (symbolized by “*”) at 0.01<P≦0.05, very significant (symbolized by “**”) at 0.001<P≦0.01, and extremely significant (symbolized by “***”) at P≦0.001. Because tests of statistical significance do not provide an estimate of the magnitude of the difference between groups, all levels of significance are described as either significant or not significant.

A scatter plot is constructed to show ΔTV values for individual animals, by group. Group mean±standard error of the mean (SEM), or median tumor volumes are plotted as linear functions of time. Group mean BW changes over the course of the study are plotted as percent change, ±SEM, from Day 1. Tumor growth curves are truncated when TR death exceeded 10%.

Results:

The following data are obtained from the study:

| | | | Mean | Statistical | | |
|---|---|---|---|---|---|---|
| | | | volume | Signif. (vs. | Mean | |
| | | | Change, | G1, G2, G3, | BW | Deaths |
| Group | n | Treatment | mm³ | G6, or G7) | Nadir | (TR/NTR) |
| 1 | 10 | Vehicle 1, | 784 | — | — | 0/0 |
| | | Vehicle 2 | (T/C = —) | | | |
| 2 | 10 | Compound I | 501 | ns (vs. G1) | — | 0/0 |
| | | (12.5 mg/kg) | (T/C = 64%) | — (others) | | |
| 3 | 10 | Compound I | 129 | *** (vs. G1) | — | 0/0 |
| | | (25 mg/kg) | (T/C = 16%) | — (others) | | |
| 4 | 7 | Compound I | 150 | ne (vs. G1) | −8.1% | 3/0 |
| | | (50 mg/kg) | (T/C = 19%) | — (others) | Day 8 | |
| 5 | 10 | RAD001 | 424 | ns (vs. G1) | — | 0/0 |
| | | (2.5 mg/kg) | (T/C = 54%) | — (others) | | |
| 6 | 10 | RAD001 | 269 | * (vs. G1) | — | 0/0 |
| | | (5 mg/kg) | (T/C = 43%) | — (others) | | |
| 7 | 10 | RAD001 | 341 | ns (vs. G1) | — | 0/0 |
| | | (10 mg/kg) | (T/C = 43%) | — (others) | | |
| 8 | 8 | Compound I | 226 | * (vs. G1) | −4.9% | 1/1 |
| | | (12/mg/kg), | (T/C = 29%) | ns (vs. G2, | Day 15 | |
| | | RAD001 | | vs. G7) | | |
| | | (10 mg/kg) | | — (others) | | |
| 9 | 8 | Compound I | 115 | *** (vs. G1) | −11.6% | 1/1 |
| | | (25 mg/kg), | (T/C = 15%) | ns (vs. G3, | Day 21 | |
| | | RAD001 | | vs. G6) | | |
| | | (10 mg/kg) | | — (others) | | |
| 10 | 0 | Compound I | — | ne (vs. G1) | −19.4% | 5/0 |
| | | (50 mg/kg), | | — (others) | Day 5 | (5 ES) |
| | | RAD001 | | | | |
| | | 2.5 mg/kg) | | | | |
| 11 | 9 | Paclitaxel | 898 | ns (using | −1.9% | 0/1 |
| | | | (T/C = 115%) | Mann-Witney | Day 12 | |
| | | | | U test) (vs. G1) | | |
| | | | | — (others) | | |

Study Endpoint = 1000 mm3; Days in Progress = 21.
n = number of animals in a group not dead from tratment-related, accidental, or unknown causes, or euthanized for sampling
Mean Volume Change = group mean volume change between Day 1 and Day 21
T/C = 100 × (ΔT/ΔC) = percent change between Day 1 and Day 21 in the mean tumor volume of a treated group (ΔT) compared with change in control group 1 (ΔC)
Statistical Significance (Kruskal-Wallis with post-hoc Dunn's multiple comparison test): ne = not evaluable, ns = not significant, * = P < 0.05; *** = P < 0.001, compared to indicated group.
Mean BW Nadir = lowest group mean body weight, as % change from Day 1 up to Day 21; — indicates no decrease in mean body weight is observed.
ES = Euthanized for sampling.

In this study, a broad range of ΔTV values for Vehicle-treated Group 1 mice results in up to 9.9-fold differences between individual animals. Significant activities are still observed with all treatments producing T/C values below the 40% threshold that denotes potential therapeutic activity.

Compound I/RAD001 combinations at the 12.5:10 and 25:5 mg/kg dose ratios (Groups 8 and 9) result in 29% and 15% T/C, and statistically significant activities (P<0.05 and P<0.001) respectively. Combination therapy at the 12.5:10 mg/kg ratio (Group 8) improves upon the Compound I and RAD001 monotherapies in Groups 2 and 7 respectively; however, the ΔTV values for Group 8 lay within the ranges of the values for Groups 2 and 7, and statistically significant improvement over the monotherapy is not observed.

Combination therapy at the 25:5 mg/kg ratio (Group 9) results in 15% T/C, and thus slightly improves upon the Compound I monotherapy in Group 3 (16% T/C). Combination of Compound I/RAD001 at the 50:2.5 mg/kg dose ratio results in 19.4% group mean BW loss on Day 5; and 50% mortality by Day 7 when the treatment is stopped.

1. A pharmaceutical combination comprising
a) a compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof, and
b) at least one mTOR inhibitor which is everolimus (RAD001), or a pharmaceutically acceptable salt thereof.

2. A pharmaceutical combination according to claim 1, wherein the mTOR inhibitor is everolimus (RAD001) or a pharmaceutically acceptable salt thereof.

3. A pharmaceutical composition comprising a pharmaceutical combination according to claim 1.

4. A method of treating a mammalian target of rapamycin (mTOR) kinase dependent diseases by administering a compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof and an one mTOR inhibitor everolimus (RAD001), or a pharmaceutically acceptable salt thereof to a warm-blooded animal in need thereof, wherein the mammalian target of rapamycin (mTOR) kinase dependent disease is breast cancer.

5. A method of treating a proliferative disease which has become resistant or has a decreased sensitivity to the treatment with at least one mTOR inhibitor selected from everolimus (RAD001), temsirolimus (CCI-779), zotarolimus (ABT578), SAR543, deferolimus (AP23573/MK-8669), AP23841, KU-0063794, INK-128, EX2044, EX3855, EX7518, AZD08055, OSI-027, WYE-125132, XL765, NV-128, WYE-125132, and EM101/LY303511 or a pharmaceutically acceptable salt thereof comprising administering a therapeutically effective amount of a compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof to a warm-blooded animal in need thereof.

6. A method according to claim 5, wherein the mTOR inhibitor is everolimus (RAD001).

7. A method according to claim 5, wherein the proliferative disease is breast cancer.
